(19) United States
(12) Patent Application Publication
(10) Pub. No.: US 2026/0000831 A1
(43) Pub. Date: Jan. 1, 2026

(54) SELF-INSERTING TROCARLESS ANALYTE-SENSING CANNULA

(71) Applicant: Pacific Diabetes Technologies Inc, Portland, OR (US)

(72) Inventors: Thomas SEIDL, Tigard, OR (US); Solomon REID, Portland, OR (US)

(21) Appl. No.: 19/268,419

(22) Filed: Jul. 14, 2025

Related U.S. Application Data
(63) Continuation of application No. PCT/US2024/011596, filed on Jan. 16, 2024.
(60) Provisional application No. 63/439,480, filed on Jan. 17, 2023.

(51) Int. Cl. A61M 5/172; A61M 5/142

(52) U.S. Cl. CPC A61M 5/1723; A61M 2005/14252; A61M 2005/1726; A61M 2230/201

(57) ABSTRACT

The disclosure describes devices and methods for insertion and delivery of insulin or an insulin analog formulation and measurement of subcutaneous glucose concentration. A device for delivery of insulin or an insulin analog formulation and measurement of subcutaneous glucose concentration, may comprise: (a) a tube comprising a proximal end and a distal end, wherein the proximal end is in fluid communication with a source of the insulin or insulin analog formulation, and wherein the distal end is configured to deliver the insulin or insulin analog formulation subcutaneously; (b) a glucose sensor disposed along a central axis of the tube; and (c) a penetrating body disposed along the central axis of the tube, wherein the penetrating body comprises a cross-sectional area no more than a cross-sectional area of the glucose sensor.

### CROSS-REFERENCE

This application is a continuation of International Application No. PCT/US2024/011596, filed Jan. 16, 2024, which claims the benefit of U.S. Provisional Application No. 63/439,480, filed Jan. 17, 2023, the contents of which are incorporated by reference in their entirety.

### STATEMENT REGARDING FEDERALLY SPONSORED RESEARCH OR DEVELOPMENT

This invention was made with government support under Contract Number 2R44DK123766-02 awarded by the National Institutes of Health (under the National Institute of Diabetes and Digestive and Kidney Diseases). The government has certain rights in the invention.

### BACKGROUND

Subjects with diabetes may be at risk of developing complications, such as kidney disease, eye disease, cardiovascular disease, and foot/nerve disease. It may be more difficult to control glucose levels in those subjects who require insulin treatment as compared to those who do not. Subjects with Type 1 Diabetes (TID) may require insulin, and many such subjects have insulin delivered using a continuous pump, which allows precise, regulated delivery of insulin 24 hours per day.

### SUMMARY

A valuable technique in managing TID is Continuous Glucose Monitoring (CGM), in which a subcutaneously inserted sensor provides interstitial glucose data to the user every few minutes. For example, JDRF-sponsored trials showed that subjects of all ages who used CGM on a regular basis experience better glycemic control than non-users (e.g., as measured by hemoglobin A1C (A1C)). However, many subjects may find CGM usage cumbersome, and many may use CGM only sporadically. Not surprisingly, when used sporadically or rarely, CGM usage may not lead to better glycemic control.

Daily life may be difficult for those who regularly use both an insulin pump and CGM. Such individuals may be required to indwell two through-the-skin devices, which may increase the risk of pain, and infection, and adversely affect device operating characteristics such as forming a seal between the subject's subcutaneous tissue and an inserted insulin delivery cannula and/or tissue damage and/or bleeding when inserting a CGM sensor that may interfere with a sensor of the CGM device.

To overcome the shortcomings of the separate CGM sensors and insulin pump devices, advances in sensor material chemistry, as described in e.g., U.S. Pat. Nos. 10,780,222 and 11,135,369, each of which is incorporated by reference herein in its entirety, have enabled the combination of the CGM sensor and insulin pump in adjacent proximity to one another and/or in a co-linear or co-axial configuration.

Although such advancements have reduced e.g., pain and infection risk of subjects, by reducing the number of insertion points for the device, there still exists an unmet need of a mechanism to assist the insertion of a CGM sensor, insulin pump cannula, and/or a combined device, to into a subject's subcutaneous tissue to ensure proper device function.

To address this unmet need, the disclosure provided herein provides devices and/or methods of inserting CGM sensors, insulin delivery cannula, and/or a combined device, to provide optimal device performance in the form of a proper seal and/or electrical connection between the CGM sensor and the surrounding subcutaneous tissue of the subject.

In an aspect, the present disclosure provides a device for delivery of insulin or an insulin analog formulation and measurement of subcutaneous glucose concentration, comprising: (a) a tube comprising a proximal end and a distal end, wherein the proximal end is in fluid communication with a source of the insulin or insulin analog formulation, and wherein the distal end is configured to deliver the insulin or insulin analog formulation subcutaneously: (b) a glucose sensor disposed along a central axis of the tube; and (c) a penetrating body disposed along the central axis of the tube, wherein the penetrating body comprises a cross-sectional area no more than a cross-sectional area of the glucose sensor.

In some embodiments, the penetrating body comprises a cross-sectional area that is less than the cross-sectional area of the glucose sensor. In some embodiments, the penetrating body comprises a cross-sectional area that is substantially equal to the cross-sectional area of the glucose sensor. In some embodiments, the cross-sectional area of the penetrating body is less than or equal to the cross-sectional area of the tube. In some embodiments, the cross-sectional area of the penetrating body is less than the cross-sectional area of the glucose sensor.

In some embodiments, the tube comprises a tapered tip at the distal end. In some embodiments, the tapered tip of the tube comprises a rounded taper or a planar taper. In some embodiments, a cross-sectional area of a distal end of the tapered of the tube is equal to the cross-sectional area of the penetrating body.

In some embodiments, the device further comprises a housing comprising an upper accessible surface and a lower surface configured to be adhered to a skin surface.

In some embodiments, the glucose sensor comprises an amperometric glucose sensor. In some embodiments, the glucose sensor is disposed on a second tube comprising a second distal end, wherein the second distal end is configured to be inserted subcutaneously.

In some embodiments, the tube comprises a taper in a direction towards the distal end of the tube. In some embodiments, the glucose sensor is disposed on a surface of the tube. In some embodiments, the glucose sensor comprises at least one electrode or at least two electrodes. In some embodiments, the at least two electrodes are electrically isolated when outside of the subject.

In some embodiments, the at least one electrode or the at least two electrodes comprise a thermoplastic material as a substrate. In some embodiments, the at least one electrode or the at least two electrodes are disposed on a surface of the penetrating body. In some embodiments, the at least one electrode or the at least two electrodes are one or more layers of the glucose sensor. In some embodiments, the at least one electrode or the at least two electrodes comprise a material of gold, carbon, graphite, platinum, or iridium. In some embodiments, the at least one electrode or the at least two electrodes are laminated to a surface of a thermoplastic substrate. In some embodiments, the surface of the thermoplastic substrate comprises at least two surfaces of the thermoplastic substrate. In some embodiments, the thermoplastic substrate is molded around the penetrating body.

In some embodiments, the glucose sensor comprises a reference electrode. In some embodiments, the reference electrode comprises a silver (Ag) or silver chloride (Ag/AgCl) reference electrode. In some embodiments, the glucose sensor further comprises an insulating layer and a metal layer, wherein the insulating layer is coupled to the metal layer, and wherein the metal layer is coupled to an electrode layer comprising the at least one electrode or the at least two electrodes. In some embodiments, the insulating layer comprises a polyimide or liquid crystal polymer. In some embodiments, the metal layer has a thickness of at least about 1 micrometer (μm), 2 μm, 3 μm, 4 μm, 5 μm, 6 μm, 7 μm, 8 μm, 9 μm, or 10 μm. In some embodiments, the metal layer comprises a material of titanium, gold, or platinum. In some embodiments, the electrode layer comprises a film having a thickness of no more than about 1000 nanometers (nm), 900 nm, 800 nm, 700 nm, 600 nm, 500 nm, 400 nm, 300 nm, 200 nm, or 100 nm. In some embodiments, a metal compound of the metal layer comprises a metal selected from the group consisting of: Osmium, Ruthenium, Palladium, Platinum, Rhodium, Iridium, Cobalt, Iron, and Copper.

In some embodiments, the penetrating body comprises a proximal and a distal end, wherein the distal end is tapered. In some embodiments, the penetrating body comprises a stylet or sharp. In some embodiments, the penetrating body comprises an inner lumen. In some embodiments, the penetrating body comprises a beveled tip.

In another aspect, the present disclosure provides a method for delivering insulin or an insulin analog formulation and measuring a subcutaneous glucose concentration, comprising: (a) providing a device for delivery of insulin or an insulin analog formulation and measurement of subcutaneous glucose concentration wherein the device comprises: (i) a tube comprising a proximal end and a distal end, wherein the proximal end is in fluid communication with a source of the insulin or insulin analog formulation, wherein the distal end is configured to deliver the insulin or insulin analog formulation subcutaneously: (ii) a glucose sensor disposed along a central axis of the tube; and (iii) a penetrating body disposed along the central axis of the tube, wherein the penetrating body comprises a cross-sectional area with no more than a cross-sectional area of the glucose sensor: (b) performing subcutaneous insertion of the distal end of the tube, the glucose sensor, and the penetrating body into a subject; and (c) delivering the insulin or the insulin analog formulation subcutaneously to the subject, or measuring a subcutaneous glucose concentration of the subject with the glucose sensor, or a combination thereof.

In some embodiments, the penetrating body comprises a cross-sectional area that is less than the cross-sectional area of the glucose sensor. In some embodiments, the penetrating body comprises a cross-sectional area that is substantially equal to the cross-sectional area of the glucose sensor. In some embodiments, the cross-sectional area of the penetrating body is less than or equal to the cross-sectional area of the tube. In some embodiments, the cross-sectional area of the penetrating body is less than the cross-sectional area of the glucose sensor.

In some embodiments, the tube comprises a tapered tip at the distal end. In some embodiments, the tapered tip of the tube comprises a rounded taper or a planar taper. In some embodiments, a cross-sectional area of a distal end of the tapered of the tube is equal to the cross-sectional area of the penetrating body.

In some embodiments, the device further comprises a housing comprising an upper accessible surface and a lower surface configured to be adhered to a skin surface.

In some embodiments, the glucose sensor comprises an amperometric glucose sensor. In some embodiments, the glucose sensor is disposed on a second tube comprising a second distal end, wherein the second distal end is configured to be inserted subcutaneously.

In some embodiments, the tube comprises a taper in a direction towards the distal end of the tube. In some embodiments, the glucose sensor is disposed on a surface of the tube. In some embodiments, the glucose sensor comprises at least one electrode or at least two electrodes. In some embodiments, the at least two electrodes are electrically isolated when outside of the subject.

In some embodiments, the at least one electrode or the at least two electrodes comprise a thermoplastic material as a substrate. In some embodiments, the at least one electrode or the at least two electrodes are disposed on a surface of the penetrating body. In some embodiments, the at least one electrode or the at least two electrodes are one or more layers of the glucose sensor. In some embodiments, the at least one electrode or the at least two electrodes comprise a material of gold, carbon, graphite, platinum, or iridium. In some embodiments, the at least one electrode or the at least two electrodes are laminated to a surface of a thermoplastic substrate. In some embodiments, the surface of the thermoplastic substrate comprises at least two surfaces of the thermoplastic substrate. In some embodiments, the thermoplastic substrate is molded around the penetrating body.

In some embodiments, the glucose sensor comprises a reference electrode. In some embodiments, the reference electrode comprises a silver (Ag) or silver chloride (Ag/AgCl) reference electrode. In some embodiments, the glucose sensor further comprises an insulating layer and a metal layer, wherein the insulating layer is coupled to the metal layer, and wherein the metal layer is coupled to an electrode layer comprising the at least one electrode or the at least two electrodes. In some embodiments, the insulating layer comprises a polyimide or liquid crystal polymer. In some embodiments, the metal layer has a thickness of at least about 1 micrometer (μm), 2 μm, 3 μm, 4 μm, 5 μm, 6 μm, 7 μm, 8 μm, 9 μm, or 10 μm. In some embodiments, the metal layer comprises a material of titanium, gold, or platinum. In some embodiments, the electrode layer comprises a film having a thickness of no more than about 1000 nanometers (nm), 900 nm, 800 nm, 700 nm, 600 nm, 500 nm, 400 nm, 300 nm, 200 nm, or 100 nm. In some embodiments, a metal compound of the metal layer comprises a metal selected from the group consisting of: Osmium, Ruthenium, Palladium, Platinum, Rhodium, Iridium, Cobalt, Iron, and Copper.

In some embodiments, the penetrating body comprises a proximal and a distal end, wherein the distal end is tapered. In some embodiments, the penetrating body comprises a stylet or sharp. In some embodiments, the penetrating body comprises an inner lumen. In some embodiments, the penetrating body comprises a beveled tip.

In another aspect, the present disclosure provides a device for delivery of insulin or an insulin analog formulation and measurement of subcutaneous glucose concentration, comprising: (a) a tube comprising a proximal end and a distal end, wherein the proximal end is in fluid communication with a source of the insulin or insulin analog formulation, and wherein the distal end is configured to deliver the insulin or insulin analog formulation subcutaneously: (b) a glucose sensor disposed along a central axis of the tube; and (c) a penetrating body disposed along the central axis of the tube, wherein the penetrating body is configured to be at least partially inserted subcutaneously without use of a trocar.

In another aspect, the present disclosure provides a method for delivering insulin or an insulin analog formulation and measuring a subcutaneous glucose concentration, comprising: (a) providing a device for delivery of insulin or an insulin analog formulation and measurement of subcutaneous glucose concentration wherein the device comprises: (i) a tube comprising a proximal end and a distal end, wherein the proximal end is in fluid communication with a source of the insulin or insulin analog formulation, wherein the distal end is configured to deliver the insulin or insulin analog formulation subcutaneously: (ii) a glucose sensor disposed along a central axis of the tube; and (iii) a penetrating body disposed along the central axis of the tube; (b) performing subcutaneous insertion of the distal end of the tube, the glucose sensor, and the penetrating body into a subject, without use of a trocar; and (c) delivering the insulin or the insulin analog formulation subcutaneously to the subject, or measuring a subcutaneous glucose concentration of the subject with the glucose sensor, or a combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a glucose sensor surrounded by a trocar that is used to penetrate a subject's skin to place the glucose sensor in the subject's subcutaneous tissue.

FIG. 2 shows a combined glucose sensor and circular and/or curved insulin delivery tube and/or cannula with a deployed penetrating member with a cross-sectional diameter less than or equal to a cross-sectional diameter of the insulin delivery tube and/or cannula.

FIGS. 3A-3B show a side perspective view of the combined glucose sensor and circular and/or curved insulin delivery tube and/or cannula with a deployed penetrating member (FIG. 3A), and a cross-sectional view of the same (FIG. 3B).

FIGS. 4A-4B show a side perspective view of the combined glucose sensor and planar insulin delivery tube and/or cannula with a deployed penetrating member (FIG. 4A), and a cross-sectional view of the same (FIG. 4B).

FIG. 5 shows a flow diagram for a method of delivering insulin or an insulin analog formulation and measuring a subcutaneous glucose concentration.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least.” “greater than.” or “greater than or equal to” precedes the first numerical value in a series of two or more numerical values, the term “at least.” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than.” “less than.” or “less than or equal to” precedes the first numerical value in a series of two or more numerical values, the term “no more than.” “less than.” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “cannula” generally refers to a hollow tube fabricated using a material, such as a polymer or a metal, having an interior (e.g., inner) surface and an exterior (e.g., outer) surface, and an opening at both ends.

As used herein, the term “sensing cannula” generally refers to a cannula having an analyte sensor (e.g., disposed on an interior surface or an exterior surface) and one or more fluid delivery channels contained within the cannula.

As used herein, the term “continuous glucose monitor (CGM)” generally refers to a system comprising electronics configured for continuous or nearly continuous measurement of glucose levels from a subject (e.g., a human being, an animal, or a mammal) and/or reporting of such measurements.

As used herein, the term “CGM infusion set” generally refers to a device (e.g., a unified device) configured for use on the skin of a subject (e.g., a human being, an animal, or a mammal) having a combination of a sensor and a cannula that includes an electrical interface to signal acquisition electronics and a port for attachment of a fluid source such as a pump or a gravity-fed sourced source.

As used herein, the term “subject.” generally refers to a person, individual, or patient. A subject can be a vertebrate, such as, for example, a mammal. Non-limiting examples of mammals include humans, simians, farm animals, sport animals, rodents, and pets. A subject may be a diabetes patient or suspected of having diabetes. The subject may be displaying a symptom(s) indicative of a health or physiological state or condition of the subject, such as diabetes. As an alternative, the subject can be asymptomatic with respect to such health or physiological state or condition.

A valuable technique in managing type 1 diabetes is Continuous Glucose Monitoring (CGM), in which a subcutaneously inserted sensor provides interstitial glucose data to the user every few minutes. For example, JDRF-sponsored trials may show that subjects of all ages who used CGM on a regular basis experience better glycemic control than non-users (e.g., as measured by hemoglobin AlC (AIC)). Insulin infusion sets for use with continuous subcutaneous insulin infusion devices (CSII, i.e., insulin pumps) and minimally invasive CGM sensors share the need to be inserted into the subcutaneous sensor through the skin prior to use.

When inserting, for example, the subcutaneous insulin infusion device, e.g., an insulin infusion cannula, a sharp and/or penetrating body, as described elsewhere herein, may be used to facilitate insertion of the insulin infusion cannula. In some cases, the insulin infusion cannula may comprise a soft and/or flexible material e.g., (silicone), or a rigid or stiff material. In some cases, the cannula may be neither sharp nor stiff enough to pierce the skin on its own without buckling. Buckling of such a plastic cannula may occur due to the forces which arise during insertion (penetration of the skin and placement in the subcutaneous tissue). Buckling may not be the only way an insertion can fail. It is possible that the sensing cannula can overcome insertion force without buckling but not insert fully into the subcutaneous tissue. Skin is flexible and may relax to close a relatively small wound, such as that made by a cannula or hypodermic needle, after the object is retracted. While the cannula remains in the skin and subcutaneous tissue, the tendency for the skin to relax may result in some pressure on the outer walls of the cannula, resulting in friction between the cannula and the surrounding tissue. This friction can result in the skin surface not relaxing to its state before insertion and remaining “puckered”. A small degree of skin puckering after insertion may resolve itself as the device is worn and small stresses due to normal body movement help the skin to relax. If the amount of puckering is too great, the skin may either not relax, or it may be painful as it does, and the device slowly damages more tissue while embedding itself more deeply.

Some characteristics affecting whether the skin remains puckered after insertion may include the energy/velocity of insertion and the friction at the outer surface of the device.

In some cases, the insulin infusion cannula may comprise a lumen configured to receive the sharp and/or penetrating body that may be deployed and/or extended to penetrate a subject's skin to allow for insertion of the insulin infusion cannula and withdrawn afterwards. Such a configuration and/or geometry of the sharp and/or penetrating body may benefit from not creating a wound in a subject's tissue larger than insulin infusion cannula itself. A wound with cross-sectional area larger than that of the cannula increases the likelihood of the inserted cannula to leak the infused fluid, e.g., insulin, back out of the wound and not be absorbed into the tissue. The possibility of the fluid leaking out from the wound site presents a serious risk to a user requiring the infused insulin to maintain their blood glucose levels since the user may not be aware of the leak and consequent decrease in dosage of insulin. Described elsewhere herein, are embodiments of a sharp and/or penetrating body that penetrates a subject's tissue with a cross-sectional area to generate an insertion point for the cannula to prevent such leaks. For successful insertion of the soft cannula, it is important that its own cross-sectional area not exceed that of the stylet at the same point where the cannula begins to enter the skin during insertion. For this reason, soft cannulae are often necked down to a smaller outer diameter at their tips. In some cases, the penetrating body may comprise a material of stainless steel. In some instances, the stainless steel may comprise 316L stainless steel. In some cases, the penetrating body may comprise a lumen and/or be hollow.

Some cannulas may comprise a soft plastic material, but it is not a requirement that a cannula be made of soft material, even in devices intended for extended use. Some users prefer to use rigid cannulae. In some cases, it may not be necessary to use an insertion aid with a cannula made of sufficiently rigid material and with sharp geometry. For example, infusion sets may use stainless steel cannulae (similar to a hypodermic needles) for delivery of insulin. Many users prefer soft plastic cannula for various reasons, but it is not a requirement that a cannula be made of soft material, even in devices intended for extended use. Some users prefer to use rigid cannulae. It is not necessary to use an insertion aid with a cannula made of sufficiently rigid material and with sharp geometry. For example, infusion sets using stainless steel cannulae (similar to hypodermic needles) for delivery of insulin exist on the market and are popular.

In the case of CGM sensors, it is generally assumed that smaller and more flexible sensors are desirable for user comfort. Since sensors are designed to be flexible, they also require an insertion aid similar to soft plastic cannula infusion sets.

As opposed to separate devices for CGM sensors and insulin infusion devices, may also be possible to create and use a combination device in which a CGM sensor is integrated into the wall of an insulin infusion cannula. As taught in U.S. Pat. Nos. 10,780,222 and 11,135,369, each of which is incorporated by reference herein in its entirety, such a combination device may require the use of a specific CGM chemistry to avoid inaccurate glucose readings caused by compounds present in insulin drug formulations. In the case of a combination CGM sensor/infusion cannula (sensing cannula), the use of a trocar is not appropriate, since the risk of medication leakage/reflux due to the larger wound cross section created by the trocar is not acceptable. If the cross-sectional area of the sensing cannula does not exceed that of a stylet placed inside it by a certain margin, then a stylet is a viable insertion aid to be used in the same manner as if the sensing cannula were simply a soft infusion cannula described above. For the cross-sectional area of the cannula material relative to its internal fluid path cross-sectional area to not exceed this margin, then the thickness of the cannula walls must be minimized.

If the walls of the cannula are thick enough that a stylet is not feasible as an insertion aid, a sufficiently sharp and rigid cannula may be inserted on its own without a separate insertion aid.

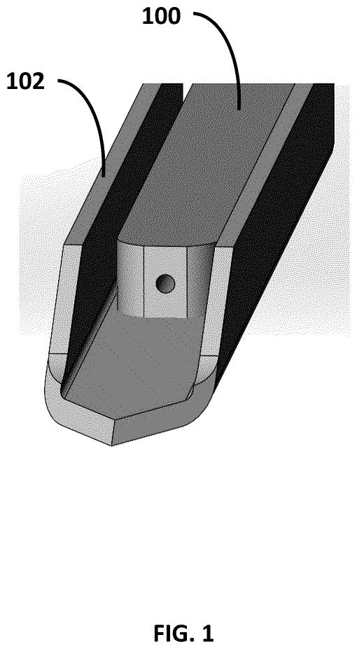

In some cases. CGM sensors 100 may comprise a cross-sectional area less than a cross-sectional area of the insulin infusion cannula and may comprise a flexible material that is desirable for user comfort when inserted. In some cases, where the CGM sensors 100 comprises a flexible material, the CGM sensor may require an insertion aid such as a sharp and/or penetrating body 102, similar to the aforementioned cannula infusion sets. Since, in some embodiments, the CGM sensor does not also serve as infusion cannula, the CGM sensor 100 may comprise a sensor that is not hollow, i.e., does not comprise a lumen. In some instances, the insertion aid may comprise a rigid sharpened structure, which pierces the skin and surrounds the flexible sensor during insertion 102, as shown in FIG. 1.

In some cases, the rigid sharpened structure may comprise a trocar. In some cases, as shown in FIG. 1, the sharp and/or penetrating body 102 may surround the CGM sensor 100, creating a wound in a subject with a larger cross-sectional area than the sensor 100 itself. The larger wound created leads to problems with excessive trauma to the subject's tissue leading to diminished accuracy of the CGM sensor that may be observed in the first hour to a day after insertion using sharp and/or penetrating bodies that exceed a cross-sectional area of the CGM sensor. Such a large diameter wound is in contrast to the case of a soft plastic infusion cannula, in which a relatively small wound may be made with a stylet of smaller diameter than the cannula. Insertion trauma caused by the sharp and/or penetrating bodies with cross-sectional areas that exceed the cross-sectional area of the CGM sensor cause excessive bleeding and the addition of cellular debris to the environment surround the CGM sensor. In some instances, the sharp and/or penetrating bodies with cross-sectional areas that exceed the cross-sectional area of the CGM sensor may cause a subsequent inflammatory response that changes the insertion region and/or location environment over the subsequent hours, affecting the response characteristics and accuracy of the sensor. In some cases, the insertion devices, and methods to insert the glucose sensor, described elsewhere herein, provide a response characteristic of a glucose sensor with at least about 75%, at least about 80%, at least about 85%, at least about 90%, at least about 95%, at least about 97%, or at least about 99% stability over up to about 2 hours from initial insertion of the glucose sensor into a subject. A sensing cannula which uses a stylet or sharp of smaller cross-sectional area than the cannula itself may minimize insertion trauma compared to if the same sensor were inserted using a trocar, likely resulting in less change in sensor response characteristics over the initial time period after insertion, ultimately enabling increased sensor accuracy during this time.

An electrochemical and/or amperometric biosensor may comprise at least two electrodes, electrically isolated from each other except during use when immersion in bodily fluid. Thermoplastic materials may be well suited to serve as the substrate for the electrodes since they typically have low electrical conductivity. Therefore, a thermoplastic cannula with electrodes on the surface may be an attractive design for a sensing cannula. A problem with thermoplastic materials is that they are not typically hard enough to perform as well as metals when they function as needles, for example when piercing the skin during insertion. A way to combine the qualities of both materials may be to surround a metal needle/sharp with electrically isolating thermoplastic material upon which the sensing electrodes can be laminated/deposited. The metal needle/sharp can be withdrawn after insertion of the sensing cannula, or it can be left in place, similar to rigid metal needle infusion cannulae.

The ability of a sensing cannula to be inserted without using an insertion aid (e.g., stylet or trocar) depends in part on the following characteristics: rigidity of the cannula/sensor material, sharpness/geometry of the penetrating tip, overall size/geometry of the cannula/sensor, lubricity/coefficient of friction of the cannula/sensor surface, and velocity/kinetic energy of insertion motion. Other factors affecting insertion success may include the insertion angle relative to the skin surface plane, axial rotation of the object during insertion, and the orientation of the sharp geometry (bevel) relative to the insertion direction.

The resistance to buckling due to insertion forces may depends on both the geometry and material properties of the cannula/sensor. Euler's critical load equation is a well-known way to determine the maximum static load that a column supports without buckling. In this case, the cannula may be thought of as a column and the skin piercing force. The critical load may depend on the stiffness of the column material, its area moment of inertia (cross sectional geometry), length, and/or the ways that the column is allowed to move at its ends. Decreased area moment of inertia, increased length, and more degrees of freedom of movement at column ends all reduce the maximum static load that a column may support.

The cannula cross section may be kept to a minimum, since insertion is generally more painful as cross-sectional area/needle diameter increases. Cannula length can be reduced up to a point, but there is a minimum length required for successful drug infusion. The cannula may be well secured where it leaves the bottom surface of the medical device housing which remains on the skin surface. The factors which may remain are the insertion velocity, cannula sharp geometry (bevel), surface friction/lubricity, and insertion angle.

Recognizing the challenges encountered through the insertion and interface of CGM sensors and/or insulin infusion cannula with a subject's skin and/or subcutaneous tissue, the present disclosure describes, in some embodiments, devices and/or methods that minimize trauma to a subject's tissue and ensure proper device interface and functionality when inserted into a subject's subcutaneous tissue.

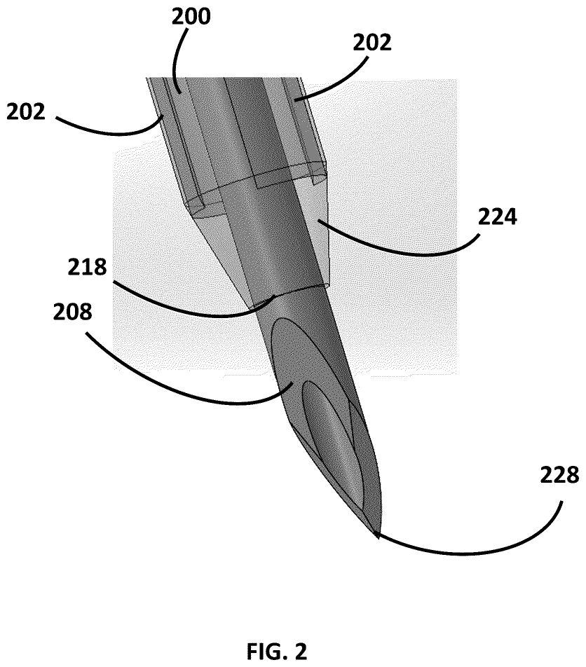

In some aspects, the disclosure provides a device for delivery of an insulin analog formulation and measurement of subcutaneous glucose concentration, as shown in FIGS. 2, 3A-3B, and 4A-4B, comprising: (a) a tube (200, 212) comprising a proximal end (214, 216) and a distal end (218, 220), where the proximal end is in fluid communication with a source of insulin and/or insulin analog formulation, where the distal end is configured to deliver the insulin and/or the insulin analog formulation subcutaneously to a subject: (b) a glucose sensor disposed along a central axis of the tube (200, 212), e.g., as shown in FIG. 2; and (c) a penetrating body 210 disposed along the central axis of the tube (200, 212), where the penetrating body 210 comprises a cross-sectional area equal to a cross-sectional area of glucose sensor. In some cases, the cross-sectional area of the penetrating body 210 may comprise a cross-sectional area less than or equal to the cross-sectional area of the tube (200, 212), as shown in FIGS. 2, 3A-3B, and 4A-4B. In some cases, the cross-sectional area of the penetrating body 210 may comprise a cross-sectional less than the cross-sectional area of the glucose sensor. In some cases, the cross-sectional area of the penetrating body less than or equal to the tube and/or the glucose sensor may provide a penetration site in a subject's skin and/or subcutaneous tissue that prevents leakage of fluid flowing through the tube and reduces bleeding and/or cellular debris from interfering with the glucose sensor reading glucose within a subcutaneous tissue of the subject, as described elsewhere herein.

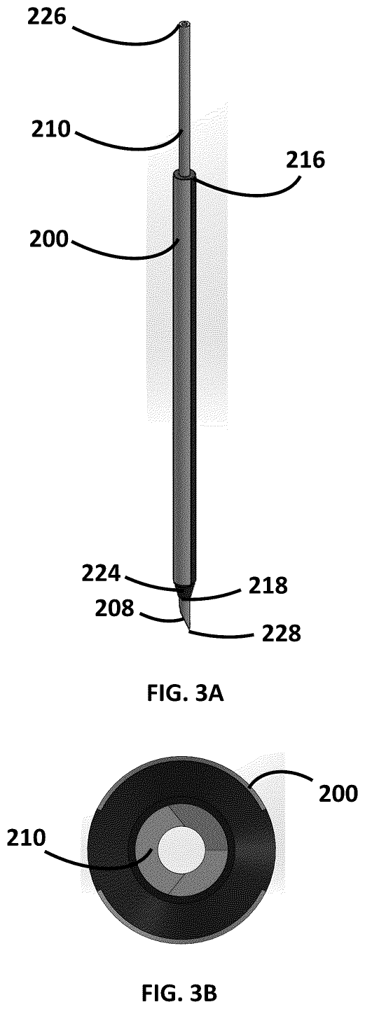

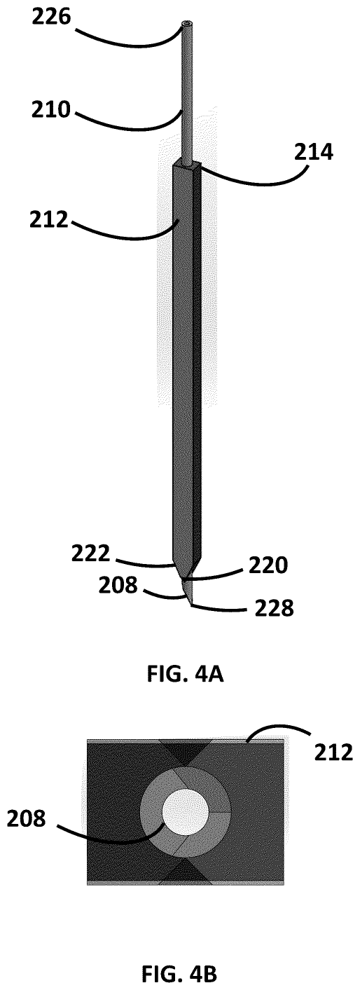

In some cases, the tube (200, 212) may comprise a taper in a direction towards the distal end of the tube (218, 220). In some cases, the tube (200, 212) and/or distal tapered tip (218, 220) may comprise a circular and/or curved geometry and/or shape (224), as shown in FIG. 3A, or a planar geometry and/or shape (222), as shown in FIG. 4A. In some cases, a cross-sectional area of a distal portion of the distal tapered tip (218, 220) may comprise a cross-sectional area less than or equal to a cross-sectional area of the penetrating body 210. In some cases, the tube (200, 212) may comprise a material of a polymer. In some instances, the tube may be molded around the penetrating body 210. In some cases, the tube (200, 212) may comprise a polymer molded component or an extruded component. In some cases, the device may further comprise a housing comprising an upper accessible surface and a lower surface configured to be adhered to a skin surface.

In some cases, the glucose sensor may comprise an amperometric glucose sensor. In some cases, the glucose sensor may be disposed on a second tube comprising a second distal end, where the second distal end is configured to be inserted subcutaneously into a subject. In some cases, the glucose sensor 202 may be disposed on a surface of the tube (200, 212), as shown in FIG. 2. The glucose sensor 202 may be disposed on an interior surface of the tube (200, 212) or an exterior surface of the tube (200, 212). In some instances, the glucose sensor 202 may comprise at least one electrode or at least two electrodes.

In some cases, the at least two electrodes of the glucose sensor may be electrically isolated from each other when outside of the subject. In some instances, the at least one electrode and/or the at least two electrodes may comprise a thermoplastic material as a substrate. In some cases, the thermoplastic material may comprise a planar thermoplastic material which planar thermoplastic material may be molded around a metal needle. In some cases, the planar thermoplastic material molded around the metal needled may be heated and conformed to a circular cross section. In some instances, the at least one electrode and/or the at least two electrodes, may be disposed on a surface of the penetrating body. In some instances, the at least one electrode and/or the at least two electrodes may at least partially surround the penetrating body.

In some cases, the at least one electrode and/or the at least two electrodes may be laminated and/or deposited on a surface of a thermoplastic deposited on a surface of the penetrating body. In some instances, the tube may comprise a thermoplastic cannula. In some cases, the thermoplastic cannula may be molded with a lumen configured to receive a metal tubing (e.g., a sharpened metal tubing), described elsewhere herein. In some cases, the at least one electrode and/or the at least two electrodes may be deposited and/or laminated on two or more surfaces of the thermoplastic cannula, after which a sharpened metal tubing may be provided to the thermoplastic cannula lumen as the penetrating body at the distal end of the thermoplastic cannula facilitating fluid path connection between the proximal and distal end of the thermoplastic cannula. In some instances, the thermoplastic cannula may be molded with a lumen, where the lumen of the thermoplastic cannula may be configured as a fluid path. In some cases, a distal tip of the thermoplastic cannula may be molded to a sharp distal end. In some cases, the at least one electrode and/or the at least two electrodes may be deposited and/or laminated on two or more surfaces of a thermoplastic material molded around a metal tubing and/or solid metal mandrel.

In some instances, after molding the thermoplastic material and laminating and/or depositing the at least one electrode and/or the at least two electrodes, the solid metal mandrel may be removed and replaced with one or more lengths of sharpened metal tubing. In some cases, the at least one electrode and/or the at least two electrodes laminated and/or deposited on a surface of a thermoplastic material may be molded around a plurality of lengths of metal tubing held straight by tension. In some cases, the at least one electrode and/or the at least two electrodes laminated and/or deposited on the surface of thermoplastic material molded on the plurality of lengths of metal tubing may be cut into a plurality of segments of a shorter length than the plurality of lengths of the metal tubing. In some cases, the plurality of segments of laminated and/or deposited electrode(s) on the thermoplastic material molded around the metal tubing may be tapered down a distal end of the thermoplastic material. In some instances, the at least one electrode and/or the at least two electrodes laminated and/or deposited on the thermoplastic material may be molded around solid and/or rounded mandrels and/or wires. After molding the at least one electrode and/or the at least two electrodes laminated and/or deposited on the thermoplastic material, the solid and/or rounded mandrels and/or wires may be removed and replaced by a sharp, e.g., a sharpened metal tubing.

In some cases, the at least one electrode and/or the at least two electrodes may comprise one or more layers of the glucose sensor. In some cases, the at least one electrode and/or the at least two electrodes may comprise a material of gold, carbon, graphite, platinum, or iridium. In some cases, the at least one electrode and/or the at least two electrodes may be laminated to a surface of a thermoplastic substrate. In some cases, the surface of the thermoplastic substrate may comprise at least two surfaces of the thermoplastic substrate. In some instances, the thermoplastic substrate may be molded around the penetrating body.

In some instances, the glucose sensor may comprise a reference electrode. In some cases, the reference electrode may comprise a material of silver (Ag) or silver chloride (AgCl). In some cases, the glucose sensor may further comprise an insulating layer and a metal layer, where the insulating layer may be coupled to the metal layer, and where the metal layer may be coupled to an electrode layer comprising the at least one electrode and/or the at least two electrodes. In some cases, the insulating layer may comprise a polyimide or liquid crystal polymer. In some cases, the metal layer may comprise a thickness of at least about 1 micrometer (μm). 2 μm, 3 μm, 4 μm, 5 μm, 6 μm, 7 μm, 8 μm, 9 μm, or 10 μm. In some cases, the metal layer may comprise a material of titanium, gold, or platinum. In some instances, the electrode layer may comprise a film of a thickness of no more than about 1000 nanometers (nm). 900 nm, 800 nm, 700 nm, 600 nm, 500 nm. 400 nm, 300 nm, 200 nm, or 100 nm. In some cases, a metal compound of the metal layer may comprise a metal selected from the group consisting of: Osmium, Ruthenium. Palladium. Platinum. Rhodium. Iridium. Cobalt. Iron, and Copper.

In some cases, the penetrating body 210 may comprise a proximal end 226 and a distal end (228). In some instances, the distal end of the penetrating body 228 may be tapered. In some cases, the penetrating body may comprise a stylet or a sharp (e.g., a needle or a stainless-steel tube cut and/or grinded to a point). In some cases, the penetrating body may comprise a beveled tip 208. In some cases, the penetrating body comprises an inner lumen and/or is hollow.

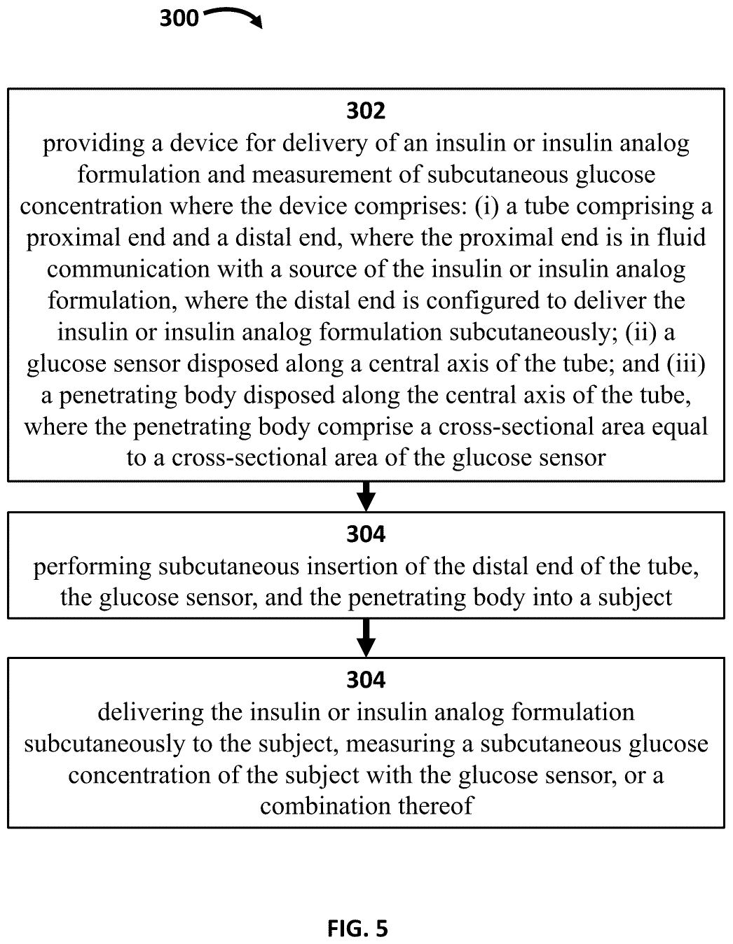

In some cases, the disclosure describes a method for delivering insulin or an insulin analog formulation and measuring a subcutaneous glucose concentration 300, as seen in FIG. 5, where the method comprises: (a) providing a device for delivery of insulin or an insulin analog formulation and measurement of subcutaneous glucose concentration where the device comprises: (i) a tube comprising a proximal end and a distal end, where the proximal end is in fluid communication with a source of the insulin or insulin analog formulation, where the distal end is configured to deliver the insulin or insulin analog formulation subcutaneously: (ii) a glucose sensor disposed along a central axis of the tube; and (iii) a penetrating body disposed along the central axis of the tube, where the penetrating body comprise a cross-sectional area equal to a cross-sectional area of the glucose sensor 302: (b) performing subcutaneous insertion of the distal end of the tube, the glucose sensor, and the penetrating body into a subject 304; and (c) delivering the insulin or insulin analog formulation subcutaneously to the subject, measuring a subcutaneous glucose concentration of the subject with the glucose sensor, or a combination thereof 304. In some cases, the insertion further comprises inserting the glucose sensor into the subject's tissue, where the subject's tissue comprises the subject's skin, epidermis, dermis, subcutaneous tissue, or any combination thereof. In some cases, the cross-sectional area of the penetrating body may comprise a cross-sectional area less than or equal to the cross-sectional area of the tube. In some cases, the cross-sectional area of the penetrating body may comprise a cross-sectional less than the cross-sectional area of the glucose sensor. In some cases, the cross-sectional area of the penetrating body less than or equal to the tube and/or the glucose sensor may provide a puncture site in a subject's skin and/or subcutaneous tissue that prevents leakage of fluid flowing through the tube and reduces bleeding and/or cellular debris from interfering with the glucose sensor reading glucose within a subcutaneous tissue of the subject, as described elsewhere herein. In some cases, the penetrating body may comprise a material of stainless steel. In some instances, the stainless steel may comprise 316L stainless steel. In some cases, the penetrating body may comprise a lumen and/or be hollow.

In some cases, the tube may comprise a taper in a direction towards the distal end of the tube. In some cases, the tube and/or distal tapered tip may comprise a circular and/or curved geometry and/or shape, or a planar geometry and/or shape. In some cases, a cross-sectional area of a distal portion of the distal tapered tip may comprise a cross-sectional area less than or equal to a cross-sectional area of the penetrating body. In some cases, the tube may comprise a material of a polymer. In some instances, the tube may be molded around the penetrating body. In some cases, the tube may comprise a polymer molded component or an extruded component. In some cases, the device may further comprise a housing comprising an upper accessible surface and a lower surface configured to be adhered to a skin surface.

In some cases, the glucose sensor may comprise an amperometric glucose sensor. In some cases, the glucose sensor may be disposed on a second tube comprising a second distal end, where the second distal end is configured to be inserted subcutaneously into a subject. In some cases, the glucose sensor may be disposed on a surface of the tube. In some instances, the glucose sensor may comprise at least one electrode or at least two electrodes. In some cases, the at least two electrodes of the glucose sensor may be electrically isolated from each other when outside the subject.

In some instances, the at least one electrode and/or the at least two electrodes may comprise a thermoplastic material as a substrate. In some cases, the thermoplastic material may comprise a planar thermoplastic material which planar thermoplastic material may be molded around a metal needle. In some cases, the planar thermoplastic material molded around the metal needled may be heated and conformed to a circular cross section.

In some instances, the at least one electrode and/or the at least two electrodes, may be disposed on a surface of the penetrating body. In some instances, the at least one electrode and/or the at least two electrodes may at least partially surround the penetrating body. In some cases, the at least one electrode and/or the at least two electrodes may be laminated and/or deposited on a surface of a thermoplastic deposited on a surface of the penetrating body.

In some cases, the at least one electrode and/or the at least two electrodes laminated and/or deposited on the surface of the thermoplastic may be inserted into the subject coupled to the penetrating body after which the penetrating body is retracted leaving the laminated and/or deposited at least one and/or the at least two electrodes in the subject.

In some cases, the at least one electrode and/or the at least two electrodes may comprise one or more layers of the glucose sensor. In some cases, the at least one electrode and/or the at least two electrodes may comprise a material of gold, carbon, graphite, platinum, or iridium. In some cases, the at least one electrode and/or the at least two electrodes may be laminated to a surface of a thermoplastic substrate. In some cases, the surface of the thermoplastic substrate may comprise at least two surfaces of the thermoplastic substrate. In some instances, the thermoplastic substrate may be molded around the penetrating body.

In some instances, the tube may comprise a thermoplastic cannula. In some cases, the thermoplastic cannula may be molded with a lumen configured to receive a metal tubing (e.g., a sharpened metal tubing), described elsewhere herein. In some cases, the at least one electrode and/or the at least two electrodes may be deposited and/or laminated on two or more surfaces of the thermoplastic cannula, after which a sharpened metal tubing may be provided to the thermoplastic cannula lumen as the penetrating body, described elsewhere herein, at the distal end of the thermoplastic cannula facilitating fluid path connection between the proximal and distal end of the thermoplastic cannula.

In some instances, the thermoplastic cannula may be molded with a lumen, where the lumen of the thermoplastic cannula may be configured as a fluid path. In some cases, a distal tip of the thermoplastic cannula may be molded to a sharp distal end. In some cases, the at least one electrode and/or the at least two electrodes may be deposited and/or laminated on two or more surfaces of a thermoplastic material molded around a metal tubing and/or solid metal mandrel. In some instances, after molding the thermoplastic material and laminating and/or depositing the at least one electrode and/or the at least two electrodes, the solid metal mandrel may be removed and replaced with one or more lengths of sharpened metal tubing.

In some cases, the at least one electrode and/or the at least two electrodes laminated and/or deposited on a surface of a thermoplastic material may be molded around a plurality of lengths of metal tubing held straight by tension. In some cases, the at least one electrode and/or the at least two electrodes laminated and/or deposited on the surface of thermoplastic material molded on the plurality of lengths of metal tubing may be cut into a plurality of segments of a shorter length than the plurality of lengths of the metal tubing. In some cases, the plurality of segments of laminated and/or deposited electrode(s) on the thermoplastic molded around the metal tubing may be tapered down a distal end of the thermoplastic material. In some instances, the at least one electrode and/or the at least two electrodes laminated and/or deposited on the thermoplastic material may be molded around solid and/or rounded mandrels and/or wires. After molding the at least one electrode and/or the at least two electrodes laminated and/or deposited on the thermoplastic material, the solid and/or rounded mandrels and/or wires may be removed and replaced by a sharp, e.g., a sharpened metal tubing.

In some instances, the glucose sensor may comprise a reference electrode. In some cases, the reference electrode may comprise a material of silver (Ag) or silver chloride (AgCl). In some cases, the glucose sensor may further comprise an insulating layer and a metal layer, where the insulating layer may be coupled to the metal layer, and where the metal layer may be coupled to an electrode layer comprising the at least one electrode and/or the at least two electrodes. In some cases, the insulating layer may comprise a polyimide or liquid crystal polymer. In some cases, the metal layer may comprise a thickness of at least about 1 micrometer (μm). 2 μm, 3 μm, 4 μm, 5 μm, 6 μm, 7 μm, 8 μm, 9 μm, or 10 μm. In some cases, the metal layer may comprise a material of titanium, gold, or platinum. In some instances, the electrode layer may comprise a film of a thickness of no more than about 1000 nanometers (nm). 900 nm, 800 nm, 700 nm, 600 nm, 500 nm. 400 nm, 300 nm, 200 nm, or 100 nm. In some cases, a metal compound of the metal layer may comprise a metal selected from the group consisting of: Osmium, Ruthenium, Palladium, Platinum, Rhodium, Iridium, Cobalt, Iron, and Copper.

In some cases, the penetrating body may comprise a proximal end and a distal end. In some instances, the distal end of the penetrating body may be tapered. In some cases, the penetrating body may comprise a stylet or a sharp (e.g., a needle or a stainless-steel tube cut and/or grinded to a point). In some cases, the penetrating body may comprise a beveled tip. In some cases, the penetrating body comprises an inner lumen and/or is hollow.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A device for delivery of insulin or an insulin analog formulation and measurement of subcutaneous glucose concentration, comprising:
(a) a tube comprising a proximal end and a distal end, wherein the proximal end is in fluid communication with a source of the insulin or insulin analog formulation, and wherein the distal end is configured to deliver the insulin or insulin analog formulation subcutaneously;
(b) a glucose sensor disposed along a central axis of the tube; and
(c) a penetrating body disposed along the central axis of the tube, wherein the penetrating body comprises a cross-sectional area no more than a cross-sectional area of the glucose sensor.

2.-34. (canceled)

35. A method for delivering insulin or an insulin analog formulation and measuring a subcutaneous glucose concentration, comprising:
(a) providing a device for delivery of insulin or an insulin analog formulation and measurement of subcutaneous glucose concentration wherein the device comprises:
(i) a tube comprising a proximal end and a distal end, wherein the proximal end is in fluid communication with a source of the insulin or insulin analog formulation, wherein the distal end is configured to deliver the insulin or insulin analog formulation subcutaneously;
(ii) a glucose sensor disposed along a central axis of the tube; and
(iii) a penetrating body disposed along the central axis of the tube, wherein the penetrating body comprises a cross-sectional area with no more than a cross-sectional area of the glucose sensor;
(b) performing subcutaneous insertion of the distal end of the tube, the glucose sensor, and the penetrating body into a subject; and
(c) delivering the insulin or the insulin analog formulation subcutaneously to the subject, or measuring a subcutaneous glucose concentration of the subject with the glucose sensor, or a combination thereof.

36. The method of claim 35, wherein the penetrating body comprises a cross-sectional area that is less than the cross-sectional area of the glucose sensor.

37. The method of claim 35, wherein the penetrating body comprises a cross-sectional area that is substantially equal to the cross-sectional area of the glucose sensor.

38. The method of claim 35, wherein the cross-sectional area of the penetrating body is less than or equal to the cross-sectional area of the tube.

39. (canceled)

40. The method of claim 35, wherein the tube comprises a tapered tip at the distal end.

41. The method of claim 35, wherein the tapered tip of the tube comprises a rounded taper or a planar taper.

42. The method of claim 35, wherein a cross-sectional area of a distal end of the tapered of the tube is equal to the cross-sectional area of the penetrating body.

43. The method of claim 35, wherein the device further comprises a housing comprising an upper accessible surface and a lower surface configured to be adhered to a skin surface.

44. The method of claim 35, wherein the glucose sensor comprises an amperometric glucose sensor.

45. The method of claim 35, wherein the glucose sensor is disposed on a second tube comprising a second distal end, wherein the second distal end is configured to be inserted subcutaneously.

46. (canceled)

47. The method of claim 35, wherein the glucose sensor is disposed on a surface of the tube.

48. The method of claim 35, wherein the glucose sensor comprises at least one electrode or at least two electrodes.

49. The method of claim 48, wherein the at least two electrodes are electrically isolated when outside the subject.

50. The method of claim 48, wherein the at least one electrode or the at least two electrodes comprise a thermoplastic material as a substrate.

51. The method of claim 48, wherein the at least one electrode or the at least two electrodes are disposed on a surface of the penetrating body.

52. The method of claim 48, wherein the at least one electrode or the at least two electrodes are one or more layers of the glucose sensor.

53. The method of claim 48, wherein the at least one electrode or the at least two electrodes comprise a material of gold, carbon, graphite, platinum, or iridium.

54. The method of claim 48, wherein the at least one electrode or the at least two electrodes are laminated to a surface of a thermoplastic substrate.

55. The method of claim 54, wherein the surface of the thermoplastic substrate comprises at least two surfaces of the thermoplastic substrate.

56. The method of claim 54, wherein the thermoplastic substrate is molded around the penetrating body.

57. The method of claim 35, wherein the glucose sensor comprises a reference electrode.

58. The method of claim 57, wherein the reference electrode comprises a silver (Ag) or silver chloride (Ag/AgCl) reference electrode.

59. The method of claim 48, wherein the glucose sensor further comprises an insulating layer and a metal layer, wherein the insulating layer is coupled to the metal layer, and wherein the metal layer is coupled to an electrode layer comprising the at least one electrode or the at least two electrodes.

60. The method of claim 59, wherein the insulating layer comprises a polyimide or liquid crystal polymer.

61. (canceled)

62. The method of claim 59, wherein the metal layer comprises a material of titanium, gold, or platinum.

63. (canceled)

64. The method of claim 59, wherein a metal compound of the metal layer comprises a metal selected from the group consisting of: Osmium, Ruthenium, Palladium, Platinum, Rhodium, Iridium, Cobalt, Iron, and Copper.

65. The method of claim 35, wherein the penetrating body comprises a proximal and a distal end, wherein the distal end is tapered.

66. The method of claim 35, wherein the penetrating body comprises one or more of a stylet or sharp, an inner lumen, or a beveled tip.

67.-69. (canceled)

70. A method for delivering insulin or an insulin analog formulation and measuring a subcutaneous glucose concentration, comprising:
(a) providing a device for delivery of insulin or an insulin analog formulation and measurement of subcutaneous glucose concentration wherein the device comprises:
(i) a tube comprising a proximal end and a distal end, wherein the proximal end is in fluid communication with a source of the insulin or insulin analog formulation, wherein the distal end is configured to deliver the insulin or insulin analog formulation subcutaneously;
(ii) a glucose sensor disposed along a central axis of the tube; and
(iii) a penetrating body disposed along the central axis of the tube;
(b) performing subcutaneous insertion of the distal end of the tube, the glucose sensor, and the penetrating body into a subject, without use of a trocar; and
(c) delivering the insulin or the insulin analog formulation subcutaneously to the subject, or measuring a subcutaneous glucose concentration of the subject with the glucose sensor, or a combination thereof.
